(19) United States
(12) Patent Application Publication
(10) Pub. No.: US 2024/0366425 A1
(43) Pub. Date: Nov. 7, 2024

(54) METHODS FOR HARVESTING, DELIVERING, AND TRANSPLANTING MACULAR TISSUE

(71) Applicant: Alcon Inc., Fribourg (CH)

(72) Inventor: Paul R. Hallen, Colleyville, TX (US)

(21) Appl. No.: 18/640,195

(22) Filed: Apr. 19, 2024

Related U.S. Application Data
(60) Provisional application No. 63/499,785, filed on May 3, 2023.

(51) Int. Cl. A61F 9/007; A61B 17/00; A61B 17/29; A61B 34/30; A61F 2/00; A61F 9/008; A61L 27/36

(52) U.S. Cl. CPC A61F 9/00727; A61B 34/30; A61F 2/0095; A61F 9/00831; A61L 27/3604; A61L 27/3641; A61B 2017/00969; A61B 17/29; A61B 2034/302; A61F 2009/00863; A61L 2430/16

(57) ABSTRACT

Described herein is a method for transplanting macular tissue. Donor macular tissue can be harvested from a donor eye, stored in a preservation vessel, and implanted into a recipient eye. Harvesting the donor macular tissue may include forming an incision around the donor macular tissue and excising the donor macular tissue. The donor macular tissue can be subsequently stored in a preservation vessel. Before implanting the donor macular tissue, diseased macular tissue may be harvested from the donor eye. Once the diseased macular tissue has been harvested, the donor macular tissue can be implanted into the recipient eye.

### BACKGROUND

Retinal degeneration is one of the leading causes of irreversible blindness worldwide, and treatment options are limited. Common to all retinal degenerative diseases is the damage or loss of photoreceptor cells of the retina. The photoreceptor cells are the light sensing cells of the retina, the delicate layer that lines the back of the eye. Photoreceptor cell loss can occur as a consequence of separation thereof from the underlying retinal pigment epithelium (RPE) and/or apoptosis. Photoreceptor cell loss leads to progressive visual impairment.

One of the most common retinal degenerative diseases is age-related macular degeneration (AMD). AMD is a deterioration of the macula, which is the central part of the retina responsible for central, high-resolution, and color vision. AMD leads to a substantial reduction in sharpness of vision and is the leading cause of visual deterioration in people over the age of 60.

Most current treatments can reduce the rate of disease progression. However, such treatments are generally not restorative and fail to completely stop photoreceptor cell loss. These treatments include antioxidant supplementation, complement inhibition using intravitreal injections, lifestyle and dietary modification, intravitreal antiangiogenic therapy, gene therapy, and implanted visual prostheses.

### SUMMARY

The present disclosure generally relates to methods and apparatus for replacing, harvesting, and delivering macular tissue.

In certain embodiments, a method for performing a macular transplant is provided. The method comprises: harvesting donor macular tissue from a donor eye; storing the donor macular tissue in a preservation vessel; and implanting the donor macular tissue into a recipient eye at a target location.

In certain embodiments, a method for harvesting macular tissue is provided. The method comprises: forming an incision around a macular tissue of an eye; excising the macular tissue from the eye; storing the macular tissue in a preservation vessel.

In certain embodiments, a method for delivering macular tissue is provided. The method comprises: forming an incision around diseased macular tissue at a target location of an eye; excising the diseased macular tissue from the eye; and implanting donor macular tissue into the eye at the target location.

### BRIEF DESCRIPTION OF THE DRAWINGS

FIG. 1A illustrates a diagram of a cross-sectional side view of an eye.

FIG. 1B illustrates an expanded or zoomed in view of the different layers of the retina at the optic nerve, where signals travel from the retina to the brain.

FIG. 2 illustrates a schematic illustration of a macular transplant, according to some embodiments herein.

FIGS. 3A-3D illustrate various operations for harvesting donor macular tissue from a donor eye, according to some embodiments herein.

FIG. 4 schematically illustrates an exemplary preservation vessel, according to some embodiments herein.

FIGS. 5A-5B illustrate operations for harvesting diseased macular tissue from a diseased eye, according to some embodiments herein.

FIGS. 5C-5F illustrate various operations for delivering donor macular tissue to a diseased eye, according to some embodiments herein.

FIG. 6 illustrates a flow diagram of a technique for performing a macular transplant, according to some embodiments herein.

FIG. 7 illustrates a flow diagram of a technique for harvesting macular tissue, according to some embodiments herein.

FIG. 8 illustrates a flow diagram of a technique for delivering macular tissue, according to some embodiments herein.

To facilitate understanding, identical reference numerals have been used, where possible, to designate identical elements that are common to the drawings. It is contemplated that elements and features of one embodiment may be beneficially incorporated in other embodiments without further recitation.

### DETAILED DESCRIPTION

It will be readily understood that the components of the embodiments as generally described herein and illustrated in the appended Figures can be arranged and designed in a wide variety of different configurations. Thus, the following more detailed description of various embodiments, as represented in the Figures, is not intended to limit the scope of the present disclosure but is merely representative of various embodiments. While the various aspects of the embodiments are presented in the Figures, the Figures are not necessarily drawn to scale unless specifically indicated.

The present disclosure may be embodied in other specific forms without departing from its spirit or essential characteristics. The described embodiments are to be considered in all respects only as illustrative and not restrictive. The scope of the present disclosure is, therefore, indicated by the appended Claims rather than by this Detailed Description. All changes which come within the meaning and range of equivalency of the Claims are to be embraced within their scope.

Reference throughout this specification to features, advantages, or similar language does not imply that all of the features and advantages that may be realized with the present disclosure should be or are in any single embodiment of the disclosure. Rather, language referring to the features and advantages is understood to mean that a specific feature, advantage, or characteristic described in connection with an embodiment is included in at least one embodiment of the present disclosure. Thus, discussions of the features and advantages, and similar language, throughout this specification may, but do not necessarily, refer to the same embodiment.

Furthermore, the described features, advantages, and characteristics of the disclosure may be combined in any suitable manner in one or more embodiments. One skilled in the relevant art will recognize, in light of the description herein, that the disclosure can be practiced without one or more of the specific features or advantages of a particular embodiment. In other instances, additional features and advantages may be recognized in certain embodiments that may not be present in all embodiments of the present disclosure.

Reference throughout this specification to “one embodiment”, “an embodiment”, or similar language means that a particular feature, structure, or characteristic described in connection with the indicated embodiment is included in at least one embodiment of the present disclosure. Thus, the phrases “in one embodiment”, “in an embodiment”, and similar language throughout this specification may, but do not necessarily, all refer to the same embodiment.

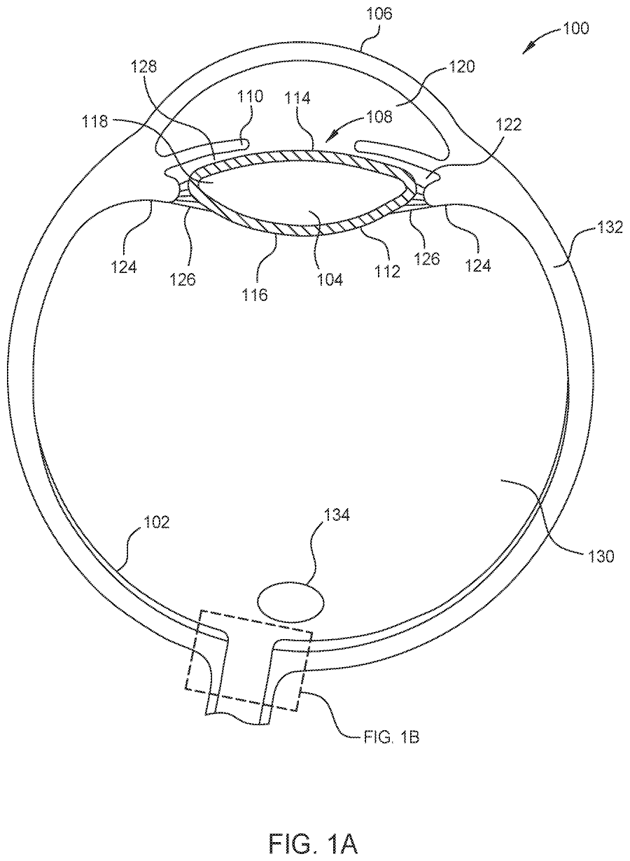

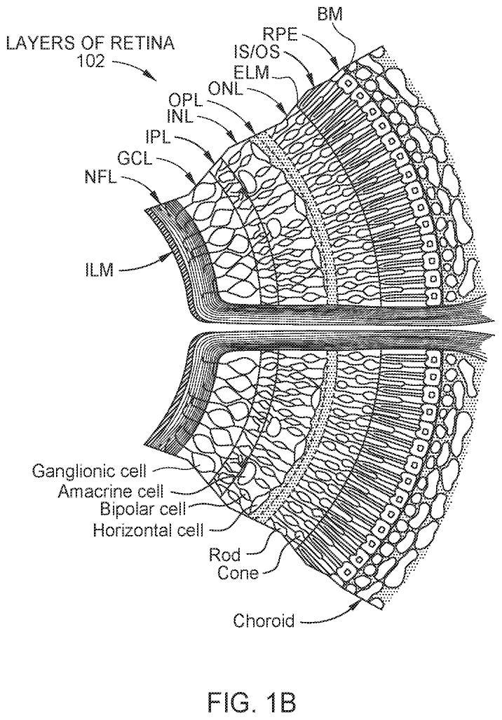

FIG. 1A illustrates a cross-sectional view of an exemplary eye 100 of a patient while FIG. 1B illustrates an expanded or zoomed in view of the different layers of a retina 102 to facilitate understanding of the present disclosure. Referring to FIG. 1A, the eye 100 includes the retina 102, a lens 104, a cornea 106, and an iris 110. A lens capsule or capsular bag 112, located behind the iris 110 of the eye 100, contains the lens 104, which is seated between an anterior capsule segment or anterior capsule 114 and a posterior capsular segment or posterior capsule 116. The anterior capsule 114 and the posterior capsule 116 meet at an equatorial region 118 of the lens capsule 112. The lens capsule 112 is attached to a ciliary body 124 via zonule fibers 126, which hold the lens capsule 112 in place. The eye 100 also includes an anterior chamber 120 located in front of the iris 110 and a posterior chamber 122 located between the iris 110 and the lens capsule 112. Behind the lens capsule 112 is an intraocular space 130 containing vitreous, which is surrounded by sclera 132.

At the back of the eye 100, the eye 100 includes the retina 102. The retina 102 is responsible for capturing light that enters the eye 100 for translation into images seen by the patient. The retina 102 generally includes a plurality of layers. Referring to FIG. 1B, layers of the retina 102 are shown from an innermost layer, the inner limiting membrane (ILM), to an outermost layer, the choroid. Between the ILM and the choroid are a retinal nerve fiber layer (NFL), a ganglion cell layer (GCL) comprising ganglionic cells, an inner plexiform layer or inner synaptic layer (IPL) and an inner nuclear layer (INL) comprising amacrine, bipolar, and horizontal cells, an outer plexiform layer or outer synaptic layer (OPL), an outer nuclear layer (ONL), an external limiting membrane (ELM), photoreceptor outer and inner segments (IS/OS) with photoreceptors (e.g., rods and cones), a retinal pigment epithelium (RPE) layer, and Bruch's membrane (BM). Generally, a full-thickness of the retina 102 may refer to all retinal layers from the ILM to the RPE, BM, and/or the choroid.

The retina 102 further includes macular tissue 134 (hereinafter referred to as “the macula 134”) at the center of the retina 102. The macula 134 is an area of the retina 102 that includes the retinal layers noted above and that is responsible for central, high-resolution, and color vision of the eye 100. The function of the macula 134 may be affected by various conditions, diseases, and/or disorders such as, increased age, trauma or structural changes to the eye, Stargardt disease (sometimes referred to as “Stargardt dystrophy” or “fundus flavimaculatus”), Best disease (sometimes referred to as “vitelliform macular dystrophy”), age-related macular degeneration (AMD), macular dystrophy, epiretinal membrane (sometimes referred to as “macular pucker”), macular holes, cystoid macular edema, vitromacular traction syndrome, and the like. The function of the macula 134 may also be affected by issues affecting the remainder of the retina. For example, retinitis pigmentosa (RP), retinal detachment, central serous chorioretinopathy (CSCR), retinal vein occlusion (RVO), posterior vitreous detachment (PVD), and the like, may affect proper macular function. Such conditions, diseases, and/or disorders that affect the macula and/or the retina may promote blurry vision, double vision (e.g., diplopia), loss of vision, eye pain, and/or light sensitivity. While some conditions, diseases, and/or disorders may be managed with surgery and/or injections, such procedures may not be restorative.

The embodiments described herein provide restorative methods for treating conditions affecting the macula, including macular diseases like AMD. The methods include implanting healthy donor macular tissue into a patient's eye where diseased macular tissue has been harvested.

Generally, donor macular tissue may be harvested from a donor eye. In some embodiments, a laser probe (e.g., femtosecond laser probe, picosecond laser probe, or other ultra-short pulse laser probe) can be used to form an incision around the donor macular tissue of the donor eye. The donor macular tissue is then excised from the donor eye using an excision instrument (e.g., forceps or a suction cannula of a suction probe). Once the donor macular tissue has been excised, the donor macular tissue is stored in a preservation vessel for subsequent transplantation into a recipient eye.

In some embodiments, the preservation vessel may include a receptacle containing a tissue storage solution and/or a biological scaffold. By storing the donor macular tissue in a preservation vessel, the donor macular tissue may be preserved for a duration of time and easily transported to, from, within, and/or between surgical sites. For example, the donor macular tissue can be transported from the donor eye to a recipient patient at the same surgical site or a different surgical site, and the harvesting and transplanting of the donor macular tissue can be carried out on the same day or different days.

Prior to transplantation of the donor macular tissue, diseased macular tissue may be harvested from the recipient eye. Generally, the same or similar methods can be used to harvest the diseased macular tissue and the donor macular tissue. In some embodiments, a laser probe (e.g., femtosecond laser probe, picosecond laser probe, or other ultra-short pulse laser probe) can be used to form an incision around the diseased macular tissue of the recipient eye. The diseased macular tissue is then excised from the recipient eye using an excision instrument (e.g., forceps or a cannula of a suction probe). Once the diseased macular tissue has been excised, donor macular tissue can be implanted into the recipient eye using a delivery instrument (e.g., forceps or a cannula of an injection device). The implanted donor macular tissue may thereafter integrate into the recipient eye and help improve vision thereof by restoring macular function(s).

Accordingly, some embodiments of the present disclosure are directed to a method of performing a macular transplant. In some embodiments, the method includes harvesting donor macular tissue from a donor eye, storing the donor macular tissue in a preservation vessel, and implanting the donor macular tissue into a recipient eye at a target location. In some embodiments, harvesting the donor macula from the donor eye includes forming an incision around the donor macular tissue of the donor eye using a laser probe, and excising the donor macular tissue from the donor eye using an excision instrument. In some embodiments, the method further includes forming an incision around diseased macular tissue at the target location of the recipient eye using a laser probe and excising the diseased macular tissue from the target location of the recipient eye using an excision instrument. In some embodiments, implanting the donor macular tissue into the recipient eye includes applying an elastomeric retinal patch over the implanted donor macular tissue. In some embodiments, a patient has been diagnosed with a macular degenerative disease and/or is in need of a macular transplant or replacement.

Some embodiments of the present disclosure are directed to a method of harvesting macular tissue. In some embodiments, the method includes forming an incision around a macular tissue of an eye, excising the macular tissue from the eye, and storing the macular tissue in a preservation vessel.

Some embodiments of the present disclosure are directed to a method of delivering macular tissue. In some embodiments, the method includes forming an incision around diseased macular tissue at a target location of an eye, excising the diseased macular tissue from the eye, and implanting donor macular tissue into the eye at the target location.

In embodiments described herein, “donor macular tissue” may refer to healthy macular tissue that may be harvested from a donor eye. A “donor eye” may refer to an eye of a donor patient (e.g., a deceased patient). As an example, the donor eye is an eye removed from a cadaver. In such an example, the donor macular tissue may or may not still be functioning when the donor macular tissue is harvested from the donor eye. Whether the donor macular tissue is functioning may be determined using an electroretinagram (ERG) test that measures an electrical response of macular tissue to photic stimulation. If the donor macular tissue is not functioning (i.e., there is minimal or no electrical response to the photic stimulation), then efforts to restore the functionality of the donor macular tissue may be made before the donor macular tissue is discarded. Efforts to restore the functionality of the donor macular tissue may include placing the donor macular tissue in a restorative solution (e.g., a nutrient-rich solutions), and/or providing electrical stimulation to the donor macular tissue. If the donor macular tissue is functioning (or functionality has been restored), then the donor macular tissue may be stored in a preservation vessel and used for macular transplant.

In embodiments described herein, “diseased macular tissue” may refer to unhealthy macular tissue that may be harvested from a recipient eye. A “recipient eye” may refer to an eye of a recipient patient (e.g., living patient). Diseased macular tissue may be macular tissue that is not functioning properly and/or that needs to be replaced. Examples of diseased macular tissue may include macular tissue that has been affected by conditions, diseases, and/or disorders which cause visual impairment, such as AMD.

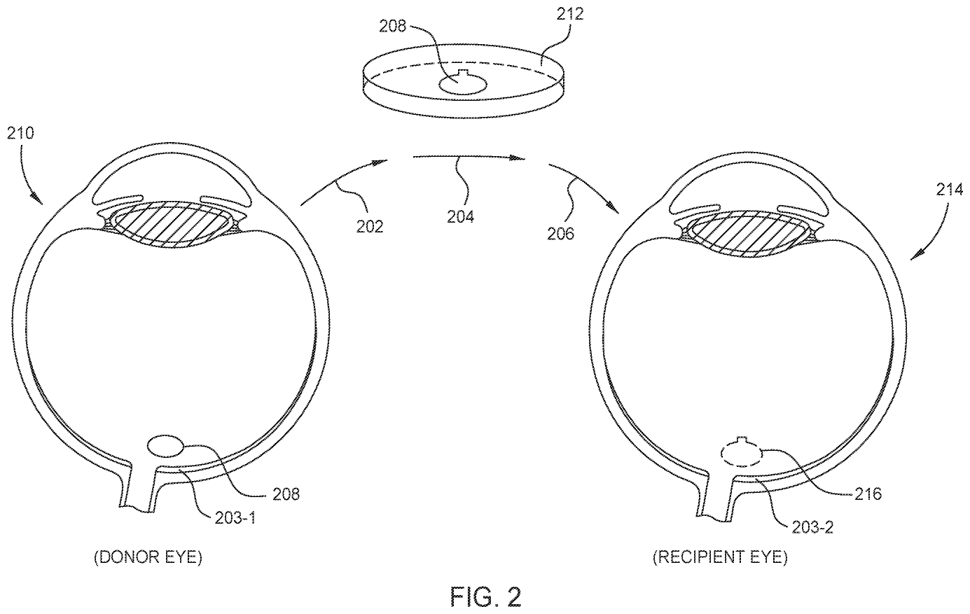

An overview of operations for performing a macular transplant is described in further detail with reference to FIG. 2.

FIG. 2 is a schematic illustration of a method for performing a macular transplant, according to some embodiments herein. As depicted in FIG. 2, performing the macular transplant includes three operations: a harvesting operation 202; a storing operation 204; and an implanting operation 206. Although FIG. 2 illustrates performing the macular transplant in three operations, the macular transplant is not limited to such operations and may involve more or less than three operations in the same or different order.

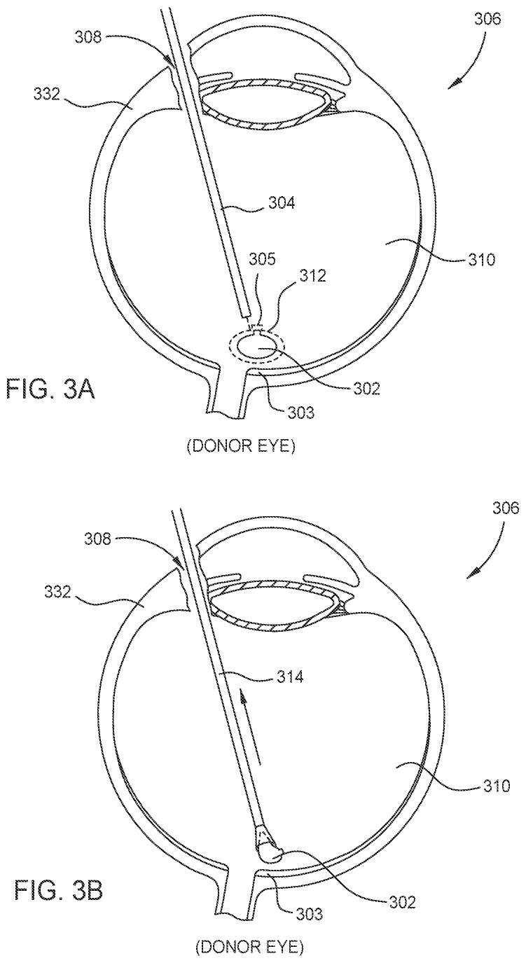

During the harvesting operation 202, donor macular tissue 208, which is part of a donor retina 203-1, is harvested from a donor eye 210. Harvesting the donor macular tissue 208 from the donor eye 210 may, in certain embodiments, include forming an incision around the donor macular tissue 208 of the donor eye 210 using a laser probe and/or other suitable cutting device, and excising the donor macular tissue 208 from the donor eye 210 using an excision instrument. The harvesting operation 202 is described in further detail below with reference to FIGS. 3A-3D.

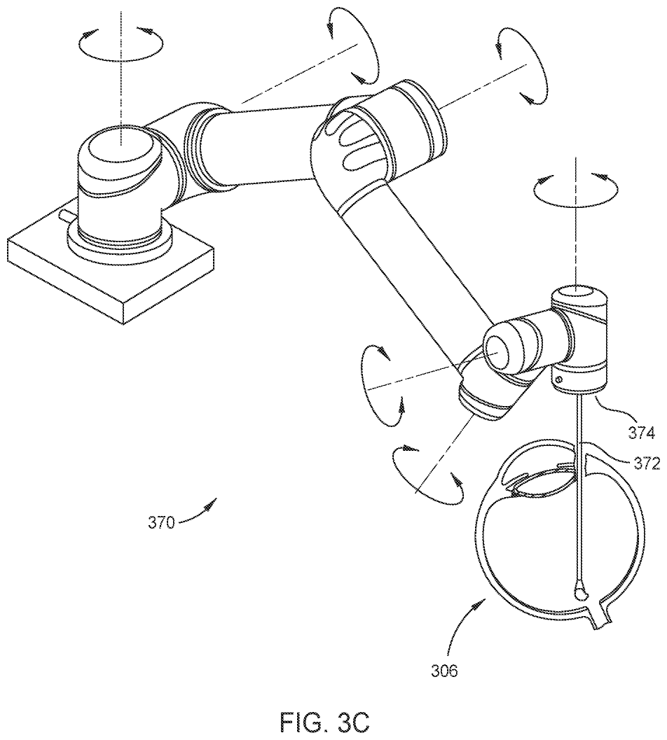

During the storing operation 204, the donor macular tissue 208 is stored in a preservation vessel 212. Storing the donor macular tissue 208 in the preservation vessel 212 may include placing the donor macular tissue 208 that has been harvested from the donor eye 210 in the preservation vessel 212. The storing operation 204 is described in further detail below with reference to FIG. 4.

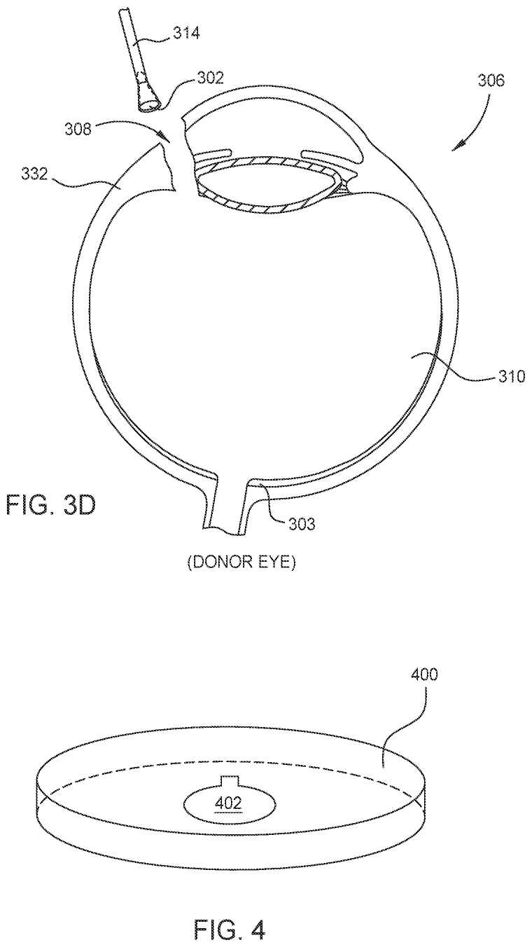

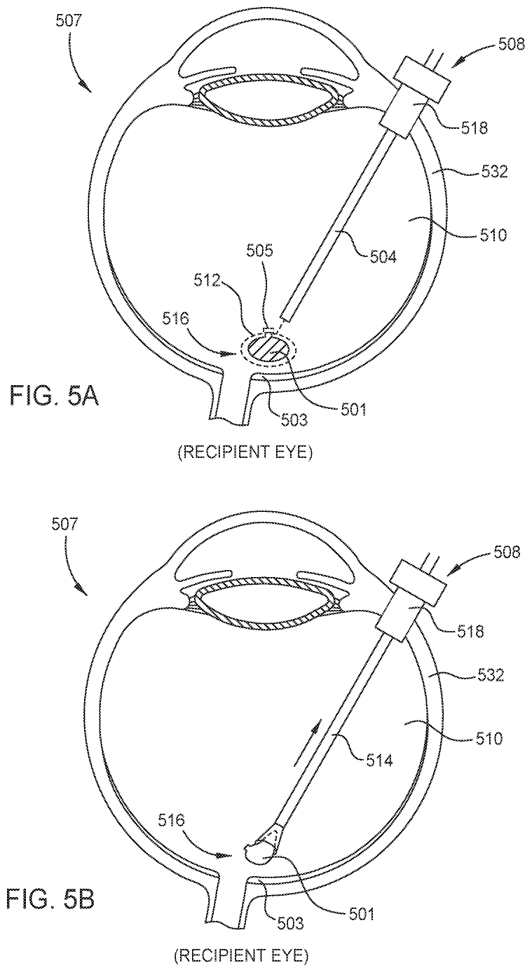

During the implanting operation 206, the donor macular tissue 208 is implanted into a recipient eye 214 at a target location 216. Before the donor macular tissue 208 is implanted into the recipient eye 214, diseased macular tissue, which is part of a recipient retina 203-2, may be harvested from the recipient eye 214. Harvesting the diseased macular tissue from the recipient eye 214 may include forming an incision around at least a portion of diseased macular tissue at the target location 216 and excising the diseased macular tissue from the target location 216 using an excision instrument. Once the diseased macular tissue has been harvested, the donor macular tissue 208 may be implanted into the recipient eye 214 in place of the diseased macular tissue. Implanting the donor macular tissue 208 into the recipient eye 214 may include implanting the donor macular tissue 208 stored in the preservation vessel 212. The implanting operation 206 is described in further detail below with reference to FIGS. 5A-5F.

Examples of steps for harvesting donor macular tissue (e.g., harvesting operation 202) are described in further detail below with reference to FIGS. 3A-3D.

FIG. 3A illustrates a first step for harvesting donor macular tissue 302, according to some embodiments herein. In FIG. 3A, a surgical cutting tool 304 is introduced into a donor eye 306 via an access point 308. In some embodiments, the surgical cutting tool 304 is introduced into the donor eye 306 via a corneal incision using an “open-sky” technique. In some embodiments, the surgical cutting tool 304 is introduced into the donor eye 306 via an access point 308 formed through the sclera 332 (e.g., a sclerotomy), pars plana (e.g., a pars plana entry), and/or the like.

The surgical cutting tool 304 is then guided through an intraocular space 310 of the donor eye 306, and an incision 312 is made around the donor macular tissue 302 in the retina 303. Forming the incision 312 around the donor macular tissue 302 may include making a fine, circular cut in the retina 303 of the donor eye 306. In certain embodiments, the incision 312 is a full-thickness incision, thereby being cut through an entire thickness of the retina 303 (e.g., from the ILM to the RPE, BM, and/or choroid) and resulting in a full-thickness graft of donor macular tissue 302 being subsequently excised from the donor eye 306. In certain embodiments, the incision 312 is a partial-thickness incision, thereby being cut through only a portion of the thickness of the retina 303 (e.g., through only a portion of the layers from the ILM to the RPE, BM, and/or choroid) and resulting in a partial-thickness graft of donor macular tissue 302 being subsequently excised from the donor eye 306.

In certain embodiments, the incision 312 around the donor macular tissue 302 may include a notch 305 or other marker on one or more sides of the donor macular tissue 302. The notch(es) 305 may be used to help orient the donor macular tissue 302 when transplanted into a recipient eye, which is described in further detail with reference to FIGS. 5A and 5D. Although a single rectangular notch is shown in FIG. 3A, the one or more notches may be triangular, circular, or any other suitable shape protruding from a main body of the donor macular tissue 302 so long as a same-shaped notch is formed in the recipient retina when harvesting the diseased macular tissue. Additionally, the notch(es) 305 may be along the perimeter on any side of the donor macular tissue 302.

In some embodiments, the surgical cutting tool 304 includes a straight blade, a round blade, scissors, or the like. In some embodiments, the surgical cutting tool 304 includes a laser probe, such as a femtosecond laser probe, a picosecond laser probe, or other ultra-short pulse laser probe. In some embodiments, the femtosecond laser probe is a “LenSx Laser” that has been modified for incising macular tissue. In such an embodiment, the femtosecond laser probe is modified to reach the retina of an eye. Using a modified femtosecond laser probe enables the use of presently available technology, which helps reduce overall costs.

Although not shown, after forming the incision 312, the surgical cutting tool 304 may be removed from the donor eye 306 via the access point 308.

FIG. 3B illustrates a second step for harvesting the donor macular tissue 302, according to some embodiments herein. In FIG. 3B, an excision instrument or other surgical tool 314 is introduced to the donor eye 306 via the access point 308 to harvest the donor macular tissue 302.

The excision instrument 314 is guided through the intraocular space 310 of the donor eye 306, and the donor macular tissue 302 is excised from the donor eye 306. In some embodiments, the excision instrument 314 may harvest an entire avascular zone of the donor macular tissue 302. A maculorhexis, capsulorhexis, or other similar excision technique may be performed to excise the donor macular tissue 302, such that a core of the donor macular tissue 302 may be harvested from the donor eye 306. The excision instrument 314 is then removed from the donor eye 306 with the donor macular tissue 302.

In some embodiments, the excision instrument 314 may include forceps (e.g., maculorhexis forceps), scissors, a suction probe (or a combined function suction and injection device) with a soft tip (e.g., silicone) cannula, a soft tip cannula (e.g., without suction functionality), a micro-dissector, or the like. In embodiments where a soft tip suction probe is used, the soft tip may comprise a funnel-like shape for facilitating the careful removal of the donor macular tissue 302 with minimal trauma thereto. In such examples, a low vacuum suction pressure may be applied, from a vacuum source fluidly coupled to the probe, to retain the donor macular tissue 302 at the soft tip of the probe as it is harvested from the donor eye 306, and to prevent deformation of the donor macular tissue 302 during its removal.

In certain embodiments, the surgical cutting tool 304 and/or excision instrument 314 may be attached to and/or operated with a robotic arm, rather than being held and/or operated directly by a surgeon. FIG. 3C illustrates an exemplary robotic arm 370 which may be implemented for excising and harvesting the donor macular tissue 302 from the donor eye 306. In FIG. 3C, the robotic arm 370 includes a tool 372 coupled at a working end 374 of the robotic arm 370. The robotic arm 370 may be part of a robotic arm system that may be used to excise and/or harvest the donor macular tissue 302.

The tool 372 may be fixed permanently at the working end 374 or may be removable and replaceable at the working end 374. The tool 372 may include any surgical instrument for excising and removing the donor macula 302, such as, for example, the surgical cutting tool 304 of FIG. 3A or the excision instrument 314 of FIG. 3B.

The configuration of the robotic arm 370 in FIG. 3C is only exemplary, and the exact configuration of the robotic arm 370 can vary considerably in any given embodiment. It may generally be preferred, though, that the robotic arm 370 include elements providing movement with at least six degrees of freedom, to facilitate effective minimally invasive macular surgical procedures, or other minimally invasive procedures, at a target location within an eye through a small incision on the eye's outer surface. An operational prototype was built using a model UR5e robotic arm supplied by Universal Robots, a robotic equipment manufacturer with its headquarters in Odense, Denmark (https://www.universal-robots.com).

Although the robotic arm 370 is described as being used for harvesting donor macular tissue 302 from a donor eye 306, the robotic arm 370 may also be used for other macular transplant steps described herein. As an example, the robotic arm 370 may be used to excise and/or implant donor macular tissue 302 from the donor eye 306 into a recipient eye. As another example, the robotic arm 370 may be used to excise and/or harvest diseased macular tissue from a recipient eye.

FIG. 3D illustrates a third step for harvesting the donor macular tissue 302, which may include a partial- or full-thickness graft of donor macular tissue 302, according to some embodiments herein. In FIG. 3D, the excision instrument 314 and the donor macular tissue 302 are guided out of and removed from the donor eye 306 via the access point 308. The donor macular tissue 302 can then be stored in a preservation vessel for preserving the donor macular tissue 302 and for transporting the donor macular tissue 302 to a recipient eye. An example of a preservation vessel for storing macular tissue is described in further detail with reference to FIG. 4.

FIG. 4 is a schematic illustration of a preservation vessel 400, according to some embodiments herein. The preservation vessel 400 may be configured to store, hold, and/or preserve macular tissue 402 prior to implantation of the macular tissue 402 into a recipient eye. In some embodiments, the preservation vessel 400 is configured to facilitate transportation of the macular tissue 402 from one surgical site to another surgical site. In some embodiments, the macular tissue 402 is the donor macular tissue 302 harvested from the donor eye 306 of FIG. 3D. In some embodiments, the macular tissue 402 is diseased macular tissue excised from a recipient eye.

The preservation vessel 400 may include any suitable type of biocompatible receptacle for storing, holding, and preserving the macular tissue 402. In some embodiments, the preservation vessel includes a petri dish. In some embodiments, the preservation vessel 400, such as a petri dish, contains a nutrient-rich tissue storage solution and/or biological scaffold for facilitating preservation of the macular tissue 402 over a duration of time. Examples of biological scaffolds include polymeric scaffolds having a biodegradable polymer material such as poly(lactic-co-glycolic acid) (PLGA), collagen, gelatin, paraleyne, polycation poly(allylanion hydrochloride) (PAH), polyanion (polyacrylic acid) (PAA), polycation poly(styrene sulfonate) (PSS), polyglycolide, poly(glycolide-co-caprolactone), poly(glycolide-co-trimethylene carbonate), polycaprolactone (PCL), polyurethane (PU), polypropylene carbonate, polyglycolic acid, polyhydroxybutyrate, polylactic acid, polydioxanone, chitosan, laminin, glycosaminoglycan, proteoglycan, heparin, elastin, fibrin, fibronectin, chondroitin sulphate proteoglycan, thiolated collagen, thiolated laminin; thiolated fibronectin, thiolated heparin, thiolated hyaluronic acid, thiolated hyaluronan-collagen-fibronectin, cellulose, hydroxyapatite, calcium phosphate, and combinations thereof. Other examples of biological scaffolds include scaffolds impregnated with cells (differentiated cells, progenitor cells, precursor cells, or combinations thereof), and scaffolds including human amniotic membrane (hAM) and/or other cellular grafts. In some embodiments, the nutrient-rich tissue storage solution and/or biological scaffold further include one or more pharmacological agents. In some embodiments, a biodegradable material scaffold or a biocompatible material (e.g., Paraleyne) may be inserted under and/or on top of the macular tissue 402 for protection prior to and/or during delivery to a recipient eye. In such an embodiment, the biodegradable material scaffold or the biocompatible material has a radius of curvature that corresponds with a diameter of the macular tissue 402.

Examples of steps for harvesting diseased macular tissue and implanting donor macular tissue are described in further detail below with reference to FIGS. 5A-5F.

FIG. 5A illustrates a first step for harvesting diseased macular tissue 501, according to some embodiments herein. The first step for harvesting the diseased macular tissue 501 may be similar to the first step for harvesting the donor macular tissue 302 described with reference to FIG. 3A.

In FIG. 5A, a surgical cutting tool 504 is introduced into a recipient eye 507 via an access point 508. The access point 508 may include an incision through the sclera 532 (e.g., a sclerotomy) and/or pars plana, or the cornea of the recipient eye 507. In some embodiments, a trocar cannula 518 may be implanted into the sclera 532 or cornea of the recipient eye 507, through which the surgical cutting tool 504 may be inserted into and/or removed from the recipient eye 507 without causing additional trauma to the tissues surrounding the access point 508.

To harvest the diseased macular tissue 501, the surgical cutting tool 504 is guided through an intraocular space 510 of the recipient eye 507, and an incision 512 is made around at least a portion of diseased macular tissue 501 at a target location 516. As an example, the target location 516 may include only a portion of the diseased macular tissue 501, or the entirety of the diseased macular tissue 501. Forming the incision 512 around the diseased macular tissue 501 may include making a fine, circular cut in the retina 503 of the recipient eye 507. In certain embodiments, the incision 512 is a full-thickness incision, thereby being cut through an entire thickness of the retina 503 (e.g., from the ILM to the RPE, BM, and/or choroid) and resulting in a full-thickness of the diseased macular tissue 501 being subsequently excised from the recipient eye 507. In certain embodiments, the incision 512 is a partial-thickness incision, thereby being cut through only a portion of the thickness of the retina 503 (e.g., through only a portion of the layers from the ILM to the RPE, BM, and/or choroid) and resulting in a partial-thickness of the diseased macular tissue 501 being subsequently excised from the recipient eye 507.

In certain embodiments, the incision 512 around the diseased macular tissue 501 may include a notch 505 or other marker on one or more sides of the diseased macular tissue 501. The notch(es) 505 may be used to help orient donor macular tissue in the recipient eye 507, as previously described with reference to FIG. 3A. Although a single rectangular notch is shown in FIG. 5A, the one or more notches may be triangular, circular, or any other suitable shape protruding from a main body of the diseased macular tissue 501 so long as a same-shaped notch is formed when harvesting the donor macular tissue. Additionally, the notch(es) 505 may be along the perimeter on any side of the diseased macular tissue 501.

In some embodiments, the surgical cutting tool 504 used to incise the diseased macular tissue 501 is the same tool used to incise donor macular tissue from a donor eye, i.e., the surgical cutting tool 504 is the surgical cutting tool 304 used to incise the donor macular tissue 302 of FIG. 3A. In some embodiments, the surgical cutting tool 504 includes a straight blade, a round blade, scissors, or the like. In some embodiments, the surgical cutting tool 504 includes a laser probe, such as a femtosecond laser probe, a picosecond laser probe, or other ultra-short pulse laser probe. In some embodiments, the femtosecond laser probe is a “LenSx Laser” that has been modified for incising macular tissue. In such an embodiment, the femtosecond laser probe is modified to reach the retina of an eye. Using a modified femtosecond laser probe enables the use of presently available technology, which helps reduce overall costs.

Although not shown, after forming the incision 512, the surgical cutting tool 504 may be removed from the recipient eye 507 via the access point 508.

FIG. 5B illustrates a second step for harvesting the diseased macular tissue 501, according to some embodiments herein. The second step for harvesting the diseased macular tissue 501 may be similar to the second step for harvesting the donor macular tissue 302 described with reference to FIG. 3B.

In FIG. 5B, an excision instrument or other surgical tool 514 is introduced to the recipient eye 507 via the access point 508 to harvest the diseased macular tissue 501. The excision instrument 514 is inserted through the trocar cannula 518 and guided through the intraocular space 510 to the diseased macular tissue 501 at the target location 516. Once the excision instrument 514 is at the target location, the diseased macular tissue 501 is excised from the recipient eye 507. A maculorhexis, capsulorhexis, or other similar excision technique may be performed to excise the diseased macular tissue 501, such that a core of the diseased macular tissue 501 may be harvested from the recipient eye 507. The excision instrument 514 is then removed from the recipient eye 507 with the diseased macular tissue 501. After the diseased macular tissue 501 has been removed from the recipient eye 507, there may be an opening, a hole, or an empty pocket left in the recipient eye 507 where the diseased macular tissue 501 has been removed. The diseased macular tissue 501 may be discarded or may be stored in another preservation vessel (different from the preservation vessel storing the donor macular tissue).

In some embodiments, the excision instrument 514 used to harvest the diseased macular tissue is the same or different excision instrument used to harvest donor macular tissue from a donor eye, i.e., the excision instrument 514 may or may not be the excision instrument 314 used to harvest the donor macular tissue 302 of FIG. 3A. For example, in some embodiments, the excision instrument 514 may include forceps (e.g., maculorhexis forceps), scissors, a suction probe (or a combined function suction and injection device), a micro-dissector, or the like. In some embodiments, the diseased macular tissue 501 may be excised using vacuum suction at the cone-like tip of the cannula. In embodiments where a soft tip suction probe is used, the soft tip may comprise a funnel-like shape for facilitating the careful removal of the diseased macular tissue 501 with minimal trauma thereto. In such examples, a low vacuum suction pressure may be applied, from a vacuum source fluidly coupled to the probe, to retain the diseased macular tissue 501 at the soft tip of the probe as it is harvested from the recipient eye 507, and to prevent deformation of the diseased macular tissue 501 during its removal.

FIG. 5C illustrates a first step for delivering donor macular tissue 502, according to some embodiments herein. The donor macular tissue 502 may be the donor macular tissue 302 that has been harvested from the donor eye 306 of FIG. 3D, and that has been stored in the preservation vessel 404 of FIG. 4. In some embodiments, the donor macular tissue 502 includes a partial-thickness graft of donor macular tissue. In some embodiments, the donor macular tissue 502 includes a full-thickness graft of donor macular tissue.

In FIG. 5C, a delivery instrument or other surgical tool 520 is used to retrieve the donor macular tissue 502 from the preservation vessel 404. The delivery instrument 520 is then introduced to the recipient eye 507 via the access point 508 to deliver the donor macular tissue 502 to the target location 516. As an example, the target location 516 is a site on the retina 503 previously occupied by the diseased macular tissue 501 of FIG. 5B (e.g., an opening, hole, or empty pocket in the retina 503). To deliver the donor macular tissue 502, the delivery instrument 520 may be inserted through the trocar cannula 518 and guided through the intraocular space 510 to the target location 516. The delivery instrument 520 may be similar to the excision instruments 314, 514, such that the delivery instrument 520 uses low vacuum suction at a cone-like tip of a suction probe cannula to retain the donor macular tissue 502 while it is delivered to the recipient eye 507, and to prevent deformation of the donor macular tissue 502 during its implantation. Delivery of the donor macular tissue 502 to the target location 516 is described in further detail with reference to FIG. 5D.

FIG. 5D illustrates a second step for delivering the donor macular tissue 502, according to some embodiments herein. In FIG. 5D, the delivery instrument 520 places the donor macular tissue 502 at the target location 516. Once the delivery instrument 520 is at the target location 516, the donor macular tissue 502 is implanted into the recipient eye 507. Implanting the donor macular tissue 502 may include placing the donor macular tissue 502 at the target location 516 or releasing the donor macular tissue 502 from the delivery instrument 520 at the target location 516. In some embodiments, the donor macular tissue 502 is placed in the hole left when the diseased macular tissue 501 was removed. Placing the donor macular tissue 502 at the target location may involve aligning the notch(es) of the donor macular tissue 502 with the notch(es) left by the diseased macular tissue 501, such that the angular orientation of the donor macular tissue 502 matches the orientation of the diseased macular tissue 501 before it was removed. When the donor macular tissue 502 is oriented correctly at the target location 516, the vacuum suction of the delivery instrument 520 may be stopped so that the donor macular tissue 502 is released from the delivery instrument 520. The delivery instrument 520 is then removed from the recipient eye 507.

Although not shown, in some embodiments, the donor macular tissue 502 may be delivered by “injecting,” or guiding, the donor macular tissue 502 from a cannula of an injection device (or combined function suction and injection device) using a plunger or other physical manipulator of the probe, or pneumatic means. For example, the plunger of an injection device may be used to push out, or dispense, the donor macular tissue 502 that has been previously aspirated into the cannula prior to delivery.

With reference to FIGS. 5A-5D, the donor macular tissue 502 and/or the diseased macular tissue 501 have a diameter between approximately 2 millimeters (mm) and 15 mm (e.g., 3-14 mm, 4-13 mm, 5-12 mm, 6-11 mm, etc.). In some embodiments, the donor macular tissue 502 and the diseased macular tissue 501 may have substantially the same lateral dimensions. For example, the donor macular tissue 502 and the diseased macular tissue 501 may have dimensions that are within at least 5 mm of each other (e.g., within 4 mm, 3 mm, 2 mm, 1 mm, or 0 mm). Substantially similar dimensions between the donor macular tissue 502 and the diseased macular tissue 501 may help facilitate more efficient integration and healing of the donor macular tissue 502 once implanted into the recipient eye 507, as well as optimal visual improvement for the patient with reduced visual disturbances.

In some embodiments, after the delivery instrument 520 has been removed from the recipient eye 507, an elastomeric retinal patch may be applied over the implanted donor macular tissue 502. An example of such an embodiment is described in further detail with reference to FIG. 5E.

FIG. 5E illustrates a third step for delivering the donor macular tissue 502, according to some embodiments herein. The third step for delivering the donor macular tissue 502 may be optional. In FIG. 5E, a retinal patch delivery instrument 522 is inserted into the recipient eye 507 through the trocar cannula 518 and guided through the intraocular space 510 to the donor macular tissue 502 at the target location 516. Once the retinal patch delivery instrument 522 is at the donor macular tissue 502, an elastomeric retinal patch 524 is applied, or injected, over the donor macular tissue 502. In some embodiments, the elastomeric retinal patch 524 may be applied in a circular motion, a snake-like motion, or other similar motion.

Generally, the retinal patch delivery instrument 522 may include any surgical instrument that is configured to deliver the elastomeric retinal patch 524. As an example, the retinal patch delivery instrument 522 may include an injection device having a cannula with a plunger that is used to push out, or dispense, the elastomeric retinal patch 524, which has been aspirated into the cannula prior to application. As another example, the retinal patch delivery instrument 522 may include forceps or similar tools with jaws to hold and place the elastomeric retinal patch 524.

The elastomeric retinal patch 524 may be applied over the implanted donor macular tissue 502 to help secure the implanted donor macular tissue 502 in place within the target location 516 of recipient eye 507 and to help prevent fluid from infiltrating a sub-retinal space of the recipient eye 507 through the hole left when the diseased macular tissue 501 was removed. The elastomeric retinal patch 524 helps secure the implanted donor macular tissue 502 within the recipient eye 507 by keeping the donor macular tissue 502 in place and preventing the donor macular tissue 502 from folding over. As an example, the elastomeric retinal patch 524 may be a biodegradable, gel-like substance that degrades after approximately two weeks from application. In some embodiments, the elastomeric retinal patch 524 includes a biodegradable polymer material, such as poly(lactic-co-glycolic acid) (PLGA), collagen, gelatin, polycation poly(allylanion hydrochloride) (PAH), polyanion (polyacrylic acid) (PAA), polycation poly(styrene sulfonate) (PSS), polyglycolide, poly(glycolide-co-caprolactone), poly(glycolide-co-trimethylene carbonate), polycaprolactone (PCL), polyurethane (PU), polypropylene carbonate, polyglycolic acid, polyhydroxybutyrate, polylactic acid, polydioxanone, chitosan, laminin, glycosaminoglycan, proteoglycan, heparin, elastin, fibrin, fibronectin, chondroitin sulphate proteoglycan, thiolated collagen, thiolated laminin; thiolated fibronectin, thiolated heparin, thiolated hyaluronic acid, thiolated hyaluronan-collagen-fibronectin, cellulose, hydroxyapatite, calcium phosphate, and combinations thereof.

FIG. 5F illustrates a fourth step for delivering the donor macular tissue 502, according to some embodiments herein. In FIG. 5F, the retinal patch delivery instrument 522 and the trocar cannula 518 of FIG. 5E have been removed from the recipient eye 507. Once the retinal patch delivery instrument 522 and the trocar cannula 518 are removed, the recipient eye 507 includes the implanted donor macular tissue 502 in place of the diseased macular tissue 501 and the elastomeric retinal patch 524 disposed over the donor macular tissue 502. A recipient patient with the recipient eye 507 that includes the implanted donor macular tissue 502 and/or the elastomeric retinal patch 524 may eventually begin to experience improved and/or restored macular function(s) as a result of the donor macular tissue 502 integrating with the recipient eye 507 and providing the macular function(s) previously lost or disrupted in the diseased macular tissue 501. Examples of improved and/or restored macular function(s) may include the improvement of fine, central, and/or color vision.

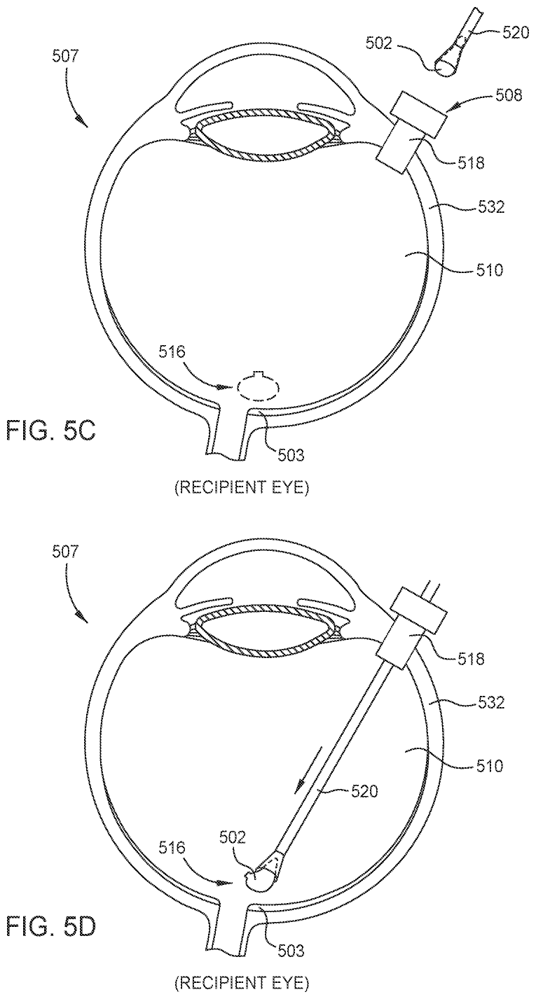

FIG. 6 illustrates a flow diagram of a technique for performing a macular transplant, according to some embodiments herein. At block 602, a donor macular tissue is harvested from a donor eye. At block 604, the donor macular tissue is stored in a preservation vessel. At block 606, the donor macular tissue is implanted into a recipient eye at a target location. In some embodiments, the target location includes a location in the retina from which diseased macular tissue was previously excised.

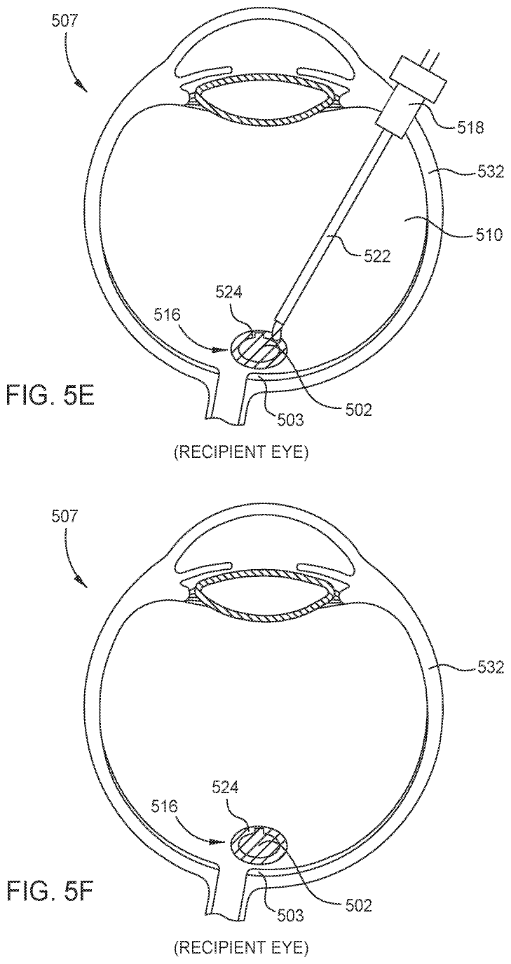

FIG. 7 illustrates a flow diagram of a technique for harvesting macular tissue, according to some embodiments herein. At block 702, an incision is formed around a macular tissue of an eye. At block 704, the macular tissue is excised from the eye. At block 706, the macular tissue is stored in a preservation vessel.

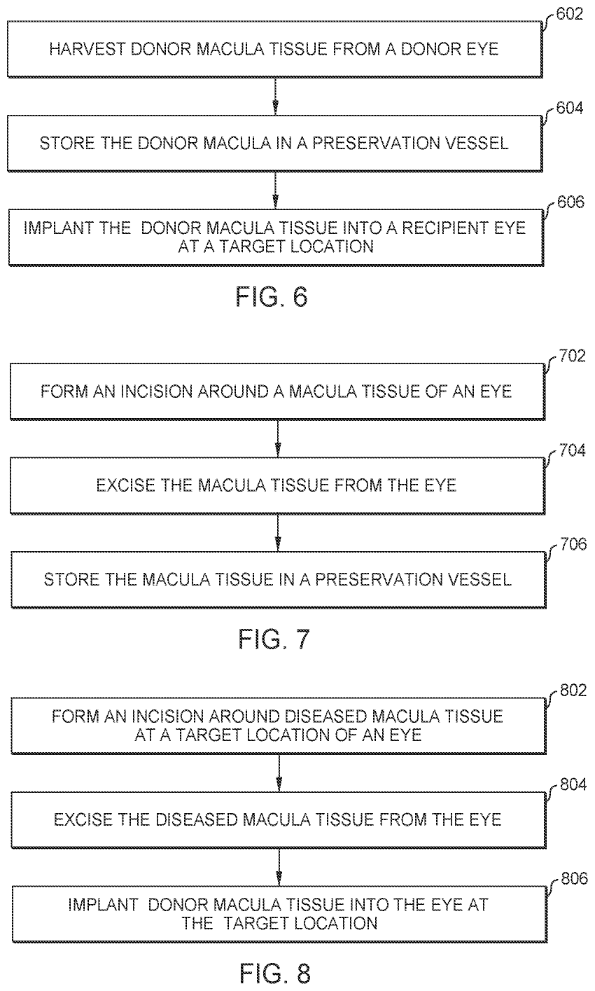

FIG. 8 illustrates a flow diagram of a technique for delivering macular tissue, according to some embodiments herein. At block 802, an incision is formed around a diseased macular tissue at a target location of an eye. At block 804, the diseased macular tissue is excised from the eye. At block 806, a donor macular tissue is implanted into the eye at the target location.

The detailed description and the drawings are supportive and descriptive of the disclosure, but the scope of the disclosure is defined solely by the claims. While some of the best modes and other embodiments for carrying out the claimed disclosure have been described in detail, various alternative designs and embodiments exist for practicing the disclosure defined in the appended claims.

Furthermore, the embodiments shown in the drawings or the characteristics of various embodiments mentioned in the present description are not necessarily to be understood as embodiments independent of each other. Rather, it is possible that each of the characteristics described in one of the examples of an embodiment can be combined with one or a plurality of other desired characteristics from other embodiments, resulting in other embodiments not described in words or by reference to the drawings. Accordingly, such other embodiments may fall within the scope of the appended claims.

What is claimed is:

1. A method for performing a macular transplant, comprising:
harvesting donor macular tissue from a donor eye;
storing the donor macular tissue in a preservation vessel; and
implanting the donor macular tissue into a recipient eye at a target location.

2. The method of claim 1, wherein harvesting the donor macula from the donor eye comprises:
forming an incision around the donor macular tissue of the donor eye using a laser probe; and
excising the donor macular tissue from the donor eye using an excision instrument.

3. The method of claim 2, wherein the laser probe comprises a femtosecond laser probe or a picosecond laser probe.

4. The method of claim 2, wherein the excision instrument comprises forceps.

5. The method of claim 1, further comprising:
forming an incision around diseased macular tissue at the target location of the recipient eye using a laser probe; and
excising the diseased macular tissue from the target location of the recipient eye using an excision instrument.

6. The method of claim 5, wherein the excised donor macular tissue and the excised diseased macular tissue have substantially the same lateral dimensions.

7. The method of claim 1, wherein implanting the donor macular tissue into the recipient eye comprises applying an elastomeric retinal patch over the implanted donor macular tissue.

8. The method of claim 7, wherein the elastomeric retinal patch is configured for:
securing the implanted donor macular tissue within the recipient eye; and
preventing fluid from infiltrating a sub-retinal space of the recipient eye.

9. The method of claim 1, wherein the donor macular tissue is at least a partial-thickness graft of macular tissue comprising photoreceptor outer and inner segments (IS/OS) and a retinal pigment epithelium (RPE) layer.

10. The method of claim 9, wherein the donor macular tissue comprises:
a full-thickness graft of macular tissue.

11. The method of claim 1, wherein the donor macular tissue comprises:
a notch for orienting the donor macular tissue in the recipient eye at the target location.

12. The method of claim 1, wherein the preservation vessel comprises a biological scaffold or a petri dish containing a tissue storage solution.

13. A method for harvesting macular tissue, comprising:
forming an incision around a macular tissue of an eye;
excising the macular tissue from the eye; and
storing the macular tissue in a preservation vessel.

14. A method for delivering macular tissue, comprising:
forming an incision around diseased macular tissue at a target location of an eye;
excising the diseased macular tissue from the eye; and
implanting donor macular tissue into the eye at the target location.
